SEQUENCE LISTING (1 sequence)

SEQ ID NO: 1 - protein, 14 aa, Mycoplasma fermentans
  MALP-2 (1)..(14): 2,3-Diacylglyceryl-cysteinyl-peptide or 2, 3-Diacylglyceral-serinyl-peptide
XGNNDESNISFKEK

(19) United States
(12) Patent Application Publication
(10) Pub. No.: US 2004/0249133 A1
(43) Pub. Date: Dec. 9, 2004

(54) Use of lipopeptides or lipoproteins for treating lung infections and lung tumours

(76) Inventors: Peter Muhlradt, Braunschweig (DE); Anke Luhrmann, Hannover (DE); Thomas Tschernig, Hildesheim (DE); Reinhard Pabst, Hannover (DE)

Correspondence Address: James P Zeller, Marshall Gerstein & Borun, 6300 Sears Tower, 233 South Wacker Drive, Chicago, IL, 60606-6357, US

(21) Appl. No.: 10/398,094

(22) PCT Filed: Oct. 2, 2001
(86) PCT No.: PCT/EP01/11414

(30) Foreign Application Priority Data
Oct. 2, 2000 (DE) 10048804.4

(51) Int. Cl. A61K 38/10

(52) U.S. Cl. 530/359; 514/14

(57) ABSTRACT

The invention relates to the use of a lipopeptide or lipoprotein having the following general structure:

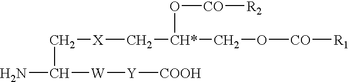

wherein:

R₁ and R₂, which may be the same or different from one another, denote C₇₋₂₅alkyl,

C₇₋₂₅alkenyl or C₇₋₂₅alkynyl,

X denotes S, O or CH₂,

W denotes CO or S(O)ₙ, (where n=1 or 2) and

Y denotes a physiologically acceptable amino acid sequence consisting of from 1 to 13 amino acid residues, and the asymmetric carbon atom labelled * has the absolute S-configuration when X=S (sulfur), for prevention of lung inflammations, for increasing lymphatic tissue in the bronchial mucosa and for treating lung infections and lung tumours.

The invention relates to the use of lipopeptides or lipoproteins of the general structure defined in claim 1 for the treatment of lung infections and lung tumours.

### INTRODUCTION

It has already been known for a relatively long time that lipopeptides which were originally isolated from micro-organisms and which are available synthetically activate macrophages (Baschang, G. 1989. Tetrahedron 45:6331-6360.). More recently, variants of such lipopeptides from mycoplasmas have become known, which by virtue of particular structural features are especially active (Mühlradt, P. F., M. Kieβ, H. Meyer, R. Süβmuth, and G. Jung. 1997. J. Exp. Med. 185:1951-1958.; Muhiradt, P. F., M. Kieβ, H. Meyer, R. Süβmuth, and G. Jung. 1998. Infect. Immun. 66: 4804-4810). Such lipopeptides are obtainable synthetically (Metzger, J. W., K.-H. Wiesmüller and G. Jung. 1991. Int. J. Peptide Protein. Res. 38: 545-554). At pg/ml concentrations in cell cultures, they are capable of stimulating various cells, including primarily macrophages, to synthesise proinflammatory cytokines (interleukin-1, interleukin-6, tumour necrosis factor) and chemokines (MIP-1, MIP-2, MCP-1, IL-8) (Deiters, U. and P. F. Mühiradt. 1999. Infect. Immun. 67: 3390-3398; Kaufmann, A., P. F. Mühlradt, D. Gemsa and H. Sprenger. 1999. Infect. Immun. 67: 6303-6308).

It is known, furthermore, that the physiological receptor of those lipopeptides is the Toll-like receptor 2 and that lipopeptides having the natural enantiomer of the lipid moiety are preferentially active (Takeuchi, A., A. Kaufmann, K. Grote, T. Kawai, K. Hoshino, M. Morr, P. F. Mühlradt and S. Akira. 2000. J. Immunol. 164: 554-557).

It has hitherto not been possible to consider using those substances therapeutically because the conventional bacterial lipoproteins were practically inactive in animal tests (Hauschildt, S., H. U. Beuscher, G. Jung, W. Bessier and A. Ulmer. 1994. FEMS Immunol. Med. Microbiol. 8:77-82). The macrophage-activating lipopeptide MALP-2 (an S-(2,3-bisacyloxypropyl)-cysteine peptide) isolated from Mycoplasma fermentans is, however, active in animal tests in the sense that intraperitoneal injection of a few μg has brought about leukocyte infiltration (Deiters, U. and P. F. Mühlradt. 1999. Infect. Immun. 67: 3390-3398).

It has hitherto not been possible, using non-toxic means, to achieve controlled infiltration of leukocytes into the lungs. That can be desirable, for example, in order to combat chronic infections or for the therapy of inoperable tumours. A corresponding therapy in the treatment of bladder tumours consists in attracting in, and activating, macrophages by injecting living mycobacteria into the bladder (Zlotta A. R, J. P. Van Vooren, 0. Denis, A. Drowart, M. Daffe, P. Lefevre, L. Schandene, M. De Cock, J. De Bruyn, P. Vandenbussche, F. Jurion, K. Palfliet, J. Simon, C. C. Schulman, J. Content, K. Huygen. 2000. Int. J. Cancer 87: 844-52). The inhalational administration of the lymphokine interleukin-2 results in the reduction of tumour metastases in the lungs with a low level of side-effects (Huland, E., H. Heinzer, H. Huland, R. Yung. 2000. Cancer J. Sci. Am. 6: Suppl.1, 104-112).

The invention is based on the surprising finding that a lipopeptide or lipoprotein having the following general structure:

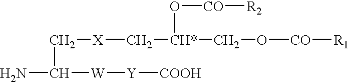

wherein:

R₁ and R₂, which may be the same or different from one another, denote C₇₋₂₅alkyl, C₇₋₂₅ alkenyl or C₇₋₂₅alkynyl,

X denotes S, O or CH₂,

W denotes CO or S(O)ₙ (where n=1 or 2) and

Y denotes a physiologically acceptable amino acid sequence consisting of from 1 to 13 amino acid residues, and the asymmetric carbon atom labelled * has the absolute S-configuration when X=S (sulfur), can be used for prevention of lung inflammations (especially in risk groups—analogously to flu protection vaccination), for increasing lymphatic tissue in the bronchial mucosa (as a result of which the effectiveness of a subsequent inhalational vaccination is improved (adjuvant effect)) and for treating lung infections and lung tumours.

In that general formula, Y can denote an amino acid sequence reading, from the N-terminal to the C-terminal end, GNNDESNISFKEK, it being possible for any 1, 2, 3, 4, 5 or 6 amino acids to be deleted or exchanged provided that the resulting lipopeptide or lipoprotein is water-soluble or amphoteric. Isofunctional amino acids, especially, may be exchanged.

For example, the amino acid sequence resulting from deletion or exchange can have a degree of homology, with respect to the starting sequence, of about 55%, especially about 60%, especially about 70%, especially about 80%, especially about 85%, especially about 90%.

In the general formula, the C₇₋₂₅alkyl, C₇₋₂₅alkenyl or C₇₋₂₅alkynyl can be a C₁₅alkyl, C₁₅alkenyl or C₁₅alkynyl, the double bond(s) in the case of a C₇₋₂₅alkenyl radical having the cis-configuration.

In the case of an S-(2,3-bisacyloxypropyl)-cysteine peptide, the acyl group is preferably a palmitoyl group.

As specific compounds there may be mentioned, for example,

S-[2,3-bispalmitoyloxy-(2RS)-propyl]cysteinyl-GNNDESNISFKEK and

S-[2,3-bispalmitoyloxy-(2S)-propyl]cysteinyl-GNNDESNISFKEK.

For use in accordance with the invention the lipopeptide or lipoprotein can be in the form of an aqueous solution, suspension or emulsion suitable for inhalation.

Use in accordance with the invention is suitable, for example, for the treatment of lung infections such as recurrent respiratory tract infections in chronic lung diseases and for the treatment of lung tumours such as primary tumours of lung epithelium and lung metastases of extrapulmonary tumours. Without intending to fix on a particular theory, it is assumed that the mechanism of action is based on the recruitment and activation of immune cells, especially on the instigation of leukocyte infiltration into the lungs. By means of the use in accordance with the invention, it is also possible, for example, for immune functions to be regained or improved after lung transplantation.

The invention will be described in greater detail hereinbelow with reference to Examples, without limitation.

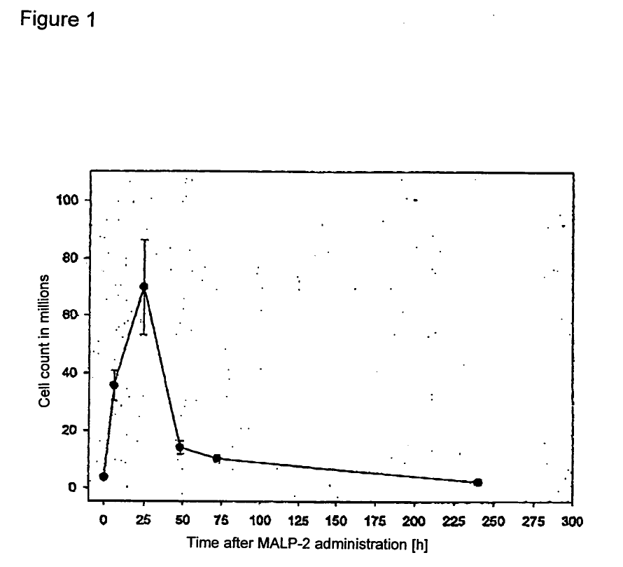

FIG. 1 shows the kinetics of the increase in the leukocyte count in bronchoalveolar lavage after administration of 2.5 μg of MALP-2.

### EXAMPLES

1) 3 μg of MALP-2 (an S-(2,3-bisacyloxypropyl)-cysteine peptide) are instilled into the trachea of rats. At various times after treatment, cells from the lung lumen are determined by means of bronchoalveolar lavage (BAL). After a few hours, the MALP-2 treatment results in an increase in the number of leukocytes in the respiratory tracts and the alveolar space of the animals, which lasts for a few days (FIG. 1). An adverse effect on the animals as a result of the MALP-2 administration was not detectable. Food consumption, bodyweight, activity and behaviour were not noteworthy.

2) With the aid of a mask, rats are made to inhale 30 μg doses of MALP-2 on six occasions at one-week intervals. This results in an increase in the lymphatic tissue in the bronchial mucosa. In this test, too, the animals were clinically not noteworthy.

1. Use of a lipopeptide or lipoprotein having the following general structure: 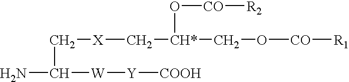
wherein:
R₁ and R₂, which may be the same or different from one another, denote C₇₋₂₅alkyl,
C₇₋₂₅alkenyl or C₇₋₂₅alkynyl,
X denotes S, O or CH₂,
W denotes CO or S(O)ₙ(where n=1 or 2) and
Y denotes a physiologically acceptable amino acid sequence consisting of from 1 to 13 amino acid residues, and the asymmetric carbon atom labelled * has the absolute S-configuration when X=S (sulfur),
for preparation of a composition for treating lung metastases of extrapulmonary tumours.

2. Use of a lipopeptide or lipoprotein having the following general structure: 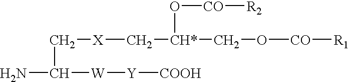
wherein:
R₁ and R₂, which may be the same or different from one another, denote C₇₋₂₅alkyl,
C₇₋₂₅alkenyl or C₇₋₂₅alkynyl,
X denotes S, O or CH₂,
W denotes CO or S(O)ₙ(where n=1 or 2) and
Y denotes a physiologically acceptable amino acid sequence consisting of from 1 to 13 amino acid residues, and the asymmetric carbon atom labelled * has the absolute S-configuration when X=S (sulfur),
for preparation of a composition for prevention of lung inflammations, for increasing lymphatic tissue in the bronchial mucosa and for treating lung infections and lung tumours except lung metastases of extrapulmonary tumours.

3. Use according to claim 1 or 2, wherein Y denotes an amino acid sequence reading, from the N-terminal to the C-terminal end, GNNDESNISFKEK, it being possible for any 1, 2, 3, 4, 5 or 6 amino acids to be deleted or exchanged provided that the resulting lipopeptide or lipoprotein is water-soluble or amphoteric.

4. Use according to claim 3, wherein the resulting amino acid sequence has a degree of homology, with respect to the starting sequence, of about 55%, especially about 60%, especially about 70%, especially about 80%, especially about 85%, especially about 90%.

5. Use according to one of the preceding claims, wherein the C₇₋₂₅alkyl, C₇₋₂₅alkenyl or C₇₋₂₅alkynyl is a C₁₅alkyl, C₁₅alkenyl or C₁₅alkynyl.

6. Use according to one of the preceding claims, wherein in the C₇₋₂₅alkenyl radical the double bond(s) have the cis-configuration.

7. Use, according to one of the preceding claims, of S-[2,3-bispalmitoyloxy-(2RS)-propyl]cysteinyl-GNNDESNISFKEK.

8. Use, according to one of claims 1 to 6, of S-[2,3-bispalmitoyloxy-(2S)-propyl]-cysteinyl-GNNDESNISFKEK.

9. Use according to one of the preceding claims, wherein the lipopeptide or lipoprotein is in the form of an aqueous solution, suspension or emulsion that is suitable for inhalation.

10. Use according to one of claims 2 to 9, wherein the lung infections are recurrent respiratory tract infections in chronic lung diseases and the lung tumours are primary tumours of lung epithelium.
